(19) United States
(12) Patent Application Publication
(10) Pub. No.: US 2026/0219413 A1
(43) Pub. Date: Jul. 30, 2026

(54) METHOD AND APPARATUS FOR MAKING IMAGING MEASUREMENTS OF A FORMATION CORE

(71) Applicant: Halliburton Energy Services, Inc., Houston, TX (US)

(72) Inventors: Baris Guner, Houston, TX (US); Ahmed Fouda, Houston, TX (US); Peter John Barrett, Houston, TX (US); Jacob Michael Proctor, Houston, TX (US)

(73) Assignee: Halliburton Energy Services, Inc., Houston, TX (US)

(21) Appl. No.: 19/041,571

(22) Filed: Jan. 30, 2025

(51) Int. Cl. G01V 3/20; E21B 47/002; G01N 27/04; G01N 33/24

(52) U.S. Cl. CPC G01V 3/20; E21B 47/002; G01N 27/041; G01N 33/24

(57) ABSTRACT

Methods and systems described herein may comprise a first container at least partially filled with a fluid; a core sample disposed within the first container and at least partially submerged within the fluid. In addition, methods and systems herein may further comprise at least one sensor from an imaging module submerged within the fluid disposed at a standoff from the core sample. The at least one sensor is configured to obtain a core sample image while the at least one sensor of the imaging module is rotated or moved around the core sample.

### BACKGROUND

Boreholes drilled into subterranean formations may enable recovery of desirable fluids (e.g., hydrocarbons) using a number of different techniques. A downhole tool may be employed in subterranean operations to determine borehole and/or formation properties.

Traditionally, borehole imager tools may be used in obtaining a detailed characterization of reservoirs. These borehole imager tools may provide a resistivity image of the formation immediately surrounding the borehole. Borehole imager tools may be used to determine formation stratigraphy, dips of the formation layers as well as, borehole and formation stress. During drilling operations borehole imager tools may be particularly important in learning about thin beds, fracture locations, and low resistivity formations. To detect thin beds, fracture locations, and low resistivity formations borehole imager may transmit a current through an injector electrode into the formation. Many other physics and tools may be employed to detect thin beds, fracture locations, and/or low resistivity formations.

Borehole imager tools exhibit complex responses. Impedance measurements of oil based mud imagers are not only a function of the formation resistivity but formation permittivity, mud resistivity, mud permittivity, borehole shape, standoff and tool geometry as well. Thus, raw apparent resistivity images from these tools may not depict the formation resistivity accurately. Applying an inversion method to decouple formation resistivity from other parameters affecting impedance measurements is one way to accomplish higher accuracy. However, inversion methods do not produce unambiguous results, and the output of the inversion may converge to a different value than the true formation resistivity. Furthermore, impedance measurements may include various measurement artifacts and noise, which may further complicate interpretation and inversion processes. As such, obtaining formation cores produce more comprehensive and accurate information on the formation properties.

Core samples are actual samples of the underground formation. Therefore, they provide valuable information in a diverse range of petrophysical and geological applications including the analysis of formation stratigraphy, detection of fractures, breakouts and washout locations, calculation of dip angles of the formation layers, and determination of the borehole and formation stress. However, taking an actual core sample from the borehole is an invasive and time-consuming procedure. Thus, cores are only available sparsely. Borehole images obtained through imager tools are used to supplement core images and/or replace them whenever they are not available. However, due to the reasons stated above, borehole images used without an actual reference may lead to wrong conclusions about the formation properties. Thus, a system and method to compare the borehole images obtained by imager tools with actual core images is needed to provide a ground truth reference for validating the performance of the imager tools, characterizing their behavior, understanding their limitations, and helping the interpretation of the images obtained downhole.

### BRIEF DESCRIPTION OF THE DRAWINGS

For a detailed description of the examples of the disclosure, reference will now be made to the accompanying drawings in which:

FIG. 1 is a schematic diagram of downhole formation downhole tool;

FIG. 2 illustrates a land-based operation;

FIG. 3 illustrates a schematic view of downhole formation downhole tool;

FIG. 4 illustrates one or more examples of obtaining a core sample;

FIG. 5 illustrates an example of an imaging analysis module;

FIG. 6 illustrates an example of pad;

FIG. 7 illustrates an example of a circuit model that may approximate the pad;

FIG. 8 is a graph illustrating real part of the measured impedance versus the formation resistivity;

FIG. 9 is a graph illustrating absolute value of the impedance versus the formation resistivity;

FIG. 10A illustrates rotating shaft example;

FIG. 10B illustrates a side view of rotating shaft example;

FIG. 11 illustrates pulley example;

FIG. 12 illustrates hydraulic arms example;

FIG. 13 illustrates a workflow for obtaining measurements.

### DETAILED DESCRIPTION

The present disclosure discloses systems and methods for obtaining an image of a core sample and characterizing and validating a downhole imaging module with the image of the core sample. In examples, systems and methods allow 360° scanning of core samples using a pad-based imager tool or another imaging module. Core sample may be submerged in a preferably pressurized fluid to enable the invasion of mud fluid into open fractures in the core. Core sample may be rotated while the imaging pad may be held at a constant position, or the imaging pad may be rotated while the core is held constant. Core sample and/or the imaging pad/imaging module may also be moved along the axis of the formation core to obtain the depth dimension of the core images. Systems and methods may also include a mount for holding the pad. Systems and methods may validate the measurements made by an imager tool, help characterize and understand the imager tool's response and provide a complementary data source to the traditional methods used in scanning of cores.

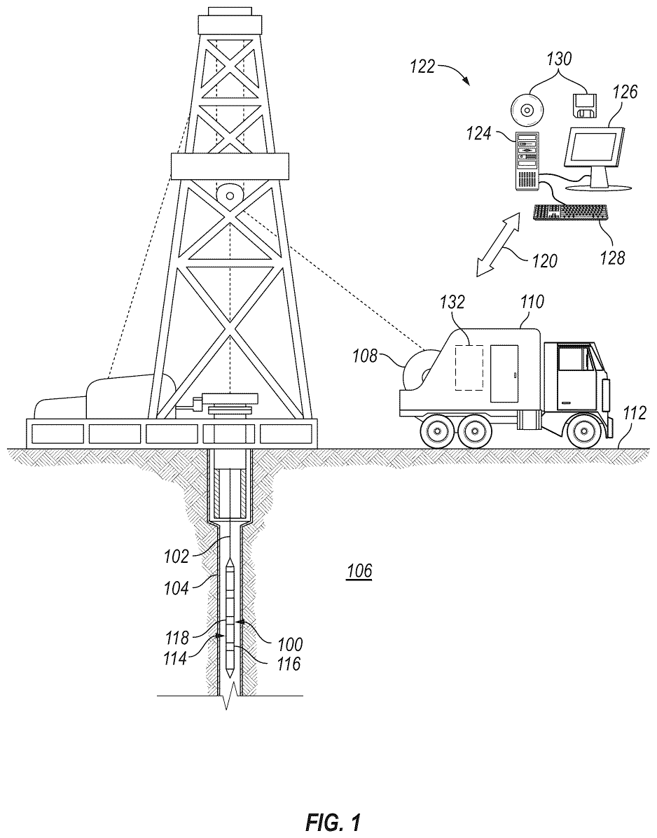

FIG. 1 is a schematic diagram of downhole tool 100 on a conveyance 102. As illustrated, wellbore 104 may extend through subterranean formation 106. While wellbore 104 is shown extending generally vertically into the subterranean formation 106, the principles described herein are also applicable to wellbores that extend at an angle through the subterranean formation 106, such as horizontal and slanted wellbores. For example, although FIG. 1 shows a vertical or low inclination angle well, high inclination angle or horizontal placement of the well and equipment is also possible. It should further be noted that while FIG. 1 generally depicts a land-based operation, those skilled in the art will readily recognize that the principles described herein are equally applicable to subsea operations that employ floating or sea-based platforms and rigs, without departing from the scope of the disclosure.

As illustrated, a hoist 108 may be used to run downhole tool 100 into wellbore 104. Hoist 108 may be disposed on a vehicle 104. Hoist 108 may be used, for example, to raise and lower conveyance 102 in wellbore 104. While hoist 108 is shown on vehicle 104, it should be understood that conveyance 102 may alternatively be disposed from a hoist 108 that is installed at surface 112 instead of being located on vehicle 104. Downhole tool 100 may be suspended in wellbore 104 on conveyance 102. Other conveyance types may be used for conveying downhole tool 100 into wellbore 104, including coiled tubing and wired drill pipe, conventional drill pipe for example. Downhole tool 100 may comprise a tool body 114, which may be elongated as shown on FIG. 1. Tool body 114 may be any suitable material, including without limitation titanium, stainless steel, alloys, plastic, combinations thereof, and the like. Downhole tool 100 may further include one or more sensors 116 for measuring properties of a core sample, a reservoir fluid, wellbore 104, subterranean formation 106, and/or the like. In examples, downhole tool 100 may also comprise imaging module 118, which may be operable to process information regarding core sample, as described below. The downhole tool 100 may be used to collect core samples from subterranean formation 106 and may obtain and separately store different core samples from subterranean formation 106.

In examples, imaging module 118 may comprise at least one sensor that may image formation 106. Such sensors include optical sensors, acoustic sensors, electromagnetic sensors, conductivity sensors, resistivity sensors, selective electrodes, density sensors, mass sensors, thermal sensors, chromatography sensors, and/or any other downhole sensors. Imaging module 118 may also be operable to determine fluid properties within the formation 106 and may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, imaging module 118 may include random access memory (RAM), one or more processing units, such as a central processing unit (CPU), or hardware or software control logic, ROM, and/or other types of nonvolatile memory.

Any suitable technique may be used for transmitting phase signals from the downhole tool 100 to surface 112. As illustrated, a communication link 120 (which may be wired or wireless, for example) may be provided that may transmit data from downhole tool 100 to an information handling system 122 at surface 112. Information handling system 122 may include a processing unit 124, a monitor 126, an input device 128 (e.g., keyboard, mouse, etc.), and/or computer media 130 (e.g., optical disks, magnetic disks) that can store code representative of the methods described herein. Information handling system 122 may act as a data acquisition system and possibly a data processing system that analyzes information from downhole tool 100. For example, information handling system 122 may process the information from downhole tool 100 for determination of fluid contamination. Information handling system 122 may also determine additional properties of the core sample. This processing may occur at surface 112 in real-time. Alternatively, the processing may occur downhole hole or at surface 112 or another location after recovery of downhole tool 100 from wellbore 104. Alternatively, the processing may be performed by an information handling system in wellbore 104, such as within one or more imaging modules 118. The resultant measurements may then be transmitted to surface 112, for example, in real-time. Real time may be defined within any range comprising 0.01 seconds to 0.1 seconds, 0.1 seconds to 1 second, 1 second to 1 minute, 1 minute to 1 hour, 1 hour to 4 hours, or any combination of ranges provided.

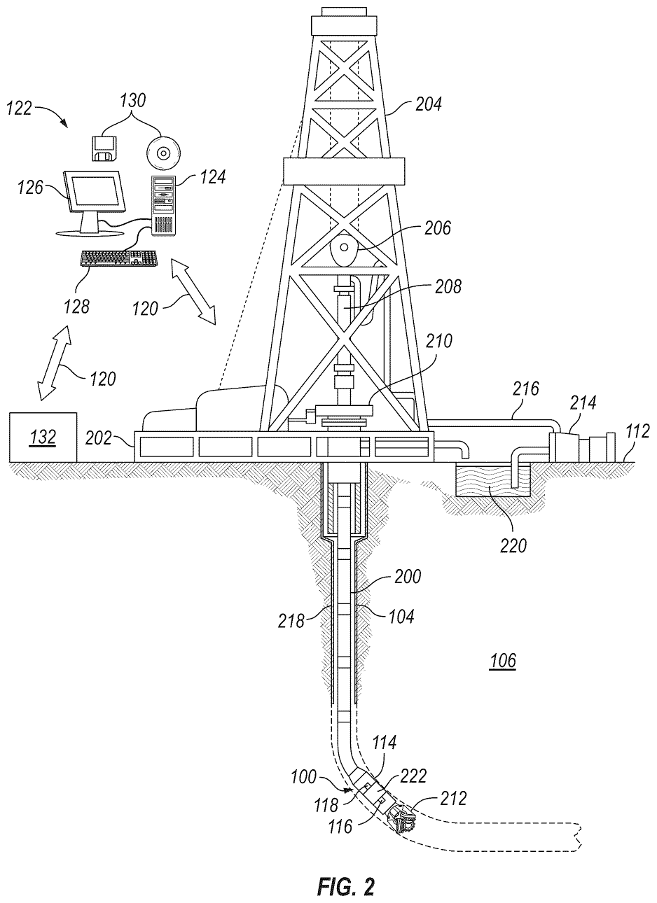

Downhole tool 100 may be used to obtain a core sample, for example, a core sample of a particular geology from subterranean formation 106. Downhole tool 100 may employ a coring bit that is deployed into the formation and drilled to a certain distance. After which, the core is broken from the formation and retrieved into the tool. Next a push rod pushes the core and deposits it into a coring tube that holds a number of cores obtained in a similar manner from different depths within the wellbore. As illustrated, a wellbore 104 may extend through subterranean formation 106. While the wellbore 104 is shown extending generally vertically into the subterranean formation 106, the principles described herein are also applicable to wellbores that extend at an angle through the subterranean formation 106, such as horizontal and slanted wellbores. For example, although FIG. 2 shows a vertical or low inclination angle well, high inclination angle or horizontal placement of the well and equipment is also possible. It should further be noted that while FIG. 2 generally depicts a land-based operation, the principles described herein are equally applicable to subsea operations that employ floating or sea-based platforms and rigs, without departing from the scope of the disclosure.

Referring now to FIG. 2, downhole tool 100 may be disposed within a drilling operation 200. As illustrated, a drilling platform 202 may support a derrick 204 having a traveling block 206 for raising and lowering drill string 200. Drill string 200 may include, but is not limited to, drill pipe and coiled tubing, as generally known to those skilled in the art. Kelly 208 may support drill string 200 as it may be lowered through a rotary table 210. A drill bit 212 may be attached to the distal end of drill string 200 and may be driven either by a downhole motor and/or via rotation of drill string 200 from the surface 112. Without limitation, drill bit 212 may include, roller cone bits, PDC bits, natural diamond bits, any hole openers, reamers, coring bits, and the like. As drill bit 212 rotates, it may create and extend wellbore 104 that penetrates various subterranean formations 106. A pump 214 may circulate drilling fluid through a feed pipe 216 to kelly 208, downhole through interior of drill string 200, through orifices in drill bit 212, back to surface 112 via annulus 218 surrounding drill string 200, and into a retention pit 220.

Drill bit 212 may be just one piece of a downhole assembly that may include one or more drill collars 222 and downhole tool 100. Downhole tool 100, which may be built into the drill collars 222 may gather measurements and core samples as described herein. One or more of the drill collars 222 may form a tool body 114, which may be elongated as shown on FIG. 2. Tool body 114 may be any suitable material, including without limitation titanium, stainless steel, alloys, plastic, combinations thereof, and the like. Downhole tool 100 may be similar in configuration and operation to downhole tool 100 shown on FIG. 1 except that FIG. 2 shows downhole tool 100 disposed on drill string 200. Alternatively, downhole tool 100 may be lowered into the wellbore after drilling operations on a wireline.

Downhole tool 100 may further include one or more sensors 116 for measuring reservoir and geologic properties of core sample, wellbore 104, subterranean formation 106, and/or the like. Downhole tool 100 may be used to collect a core sample from subterranean formation 106. Downhole tool 100 may obtain and separately store different core samples from subterranean formation 106 with one or more imaging modules 118. Imaging modules 118 may operate and function in the same manner as described above.

As previously described, information from downhole tool 100 may be transmitted to an information handling system 122, which may be located at surface 112. As illustrated, communication link 120 (which may be wired or wireless, for example) may be provided that may transmit data from downhole tool 100 to an information handling system 111 at surface 112. Information handling system 122 may include a processing unit 124, a monitor 126, an input device 128 (e.g., keyboard, mouse, etc.), and/or computer media 130 (e.g., optical disks, magnetic disks) that may store code representative of the methods described herein. In addition to processing on downhole tool 100, processing with imaging module 118 may occur at surface 112, to be discussed in detail below. In examples, information handling system 122 may perform computations to derive geological and reservoir properties.

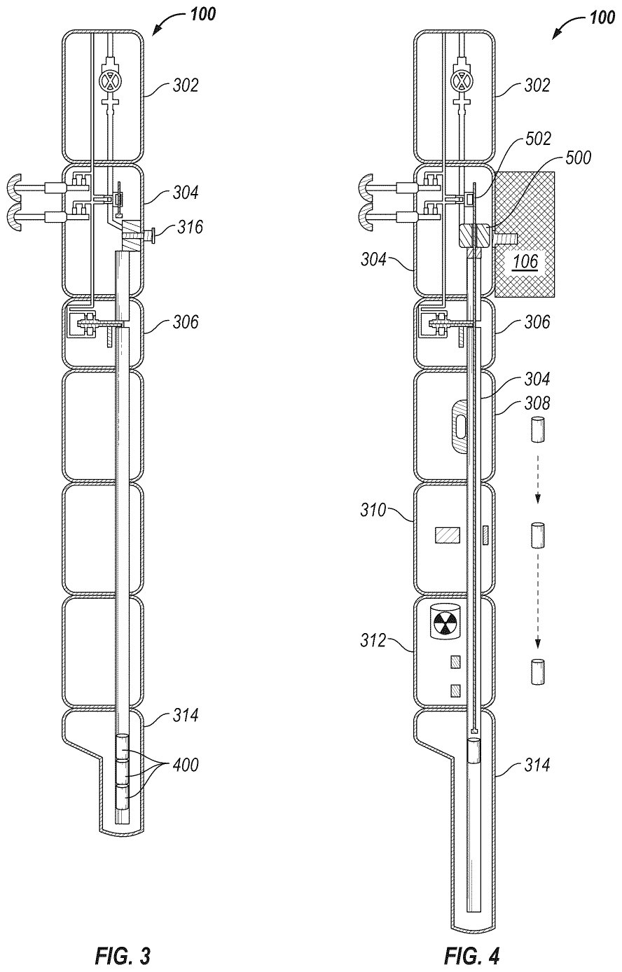

FIG. 3 illustrates a schematic view of downhole tool 100. As illustrated, downhole tool 100 may include one or more modules. For example, modules may comprise, but are not limited to, a motor module 302, a coring module 304, a core marker module 306, and/or a core storage module 314. As will be discussed in further detail below, distances between each module may be utilized for identifying one or more properties of a core sample. In examples, side drill 316 may drill into formation 106 (e.g. referring to FIG. 1) and extract core sample 400. Once extracted, core sample 400 may move into coring module 304 and core tube 404 via a push rod 402 (e.g., referring to FIG. 1). Core sample 400 may be transported to core marker module 306, and/or a core storage module 314 via core tube 404. Herein, a module is defined as a distinct housing that provides a structural support for one or more sensor, devices, and/or the like that may aid in measuring properties of a medium such as a core sample or any part of a subterranean formation. Additionally, a module may connect to other modules to form downhole tool 100. Additionally, a module may comprise a walled shell that form the outer area of each housing. Within the walled shell are the structural supports that may connect the one or more sensors, devices, and/or the like to the module. In examples, each module may have one or more individual pipes in which may allow the core sample 400 may move through the module. As discussed in further detail below, each module may connect to another module and one module may be exchanged and replaced with a different module.

FIG. 4 illustrates one or more examples of obtaining a core sample 400 during measurement operations. As illustrated, within downhole tool 100, coring module 304 may remove a core sample 400 from subterranean formation 106 into an individual tube. The individual tube may form a segment of core tube 404. Disposed within coring module 304, push rod 402 may be configured to transport core sample 400 at any selected speed through the channel formed from the core tube 404. Push rod 402 may be controlled by and connected to one or more devices within a motor module 302. Push rod 402 is controlled electro-mechanically. Motor module 302 may include a hydraulic line connected to the push rod 402 through relays. The relays may control the functions “Rod Extend” and “Rod Retract”. Multiple push rods 402 may be comprised for each relay. When the Operator toggles either relay, it may be actuated and increase the hydraulic pressure pushing push rod 402 down or deplete the hydraulic pressure, retracting the rod to its original position.

Additionally, core tube 404 may traverse through coring module 304, core marker module 306, core storage module 314, and/or any module that may comprise downhole tool 100. Obtaining core sample 400 may be performed in a wireline or drilling implementation. Core storage module 314 comprises an individual tube which may store core samples 400. Core samples 400 may be stored within core storage module until downhole tool 100 is returned to the surface. Core storage module 314 may be a pressurized vessel to ensure the core sample remains saturated with original formation fluid, to be discussed below. FIGS. 3 and 4 illustrate an example of downhole tool 100 with an emphasis on the coring mechanism. FIGS. 3 and 4 illustrate rotary coring, however, any other form of coring may be a suitable technique for obtaining core sample 400. For example, percussion coring comprising hollow, retrievable bullets shot into the formation with a gun on the wireline may obtain core sample 400. In addition, imaging module 118 may be disposed on downhole tool 100. Further, the examples provided below illustrate imaging module 118 disposed without downhole tool 100 in wellbore 104.

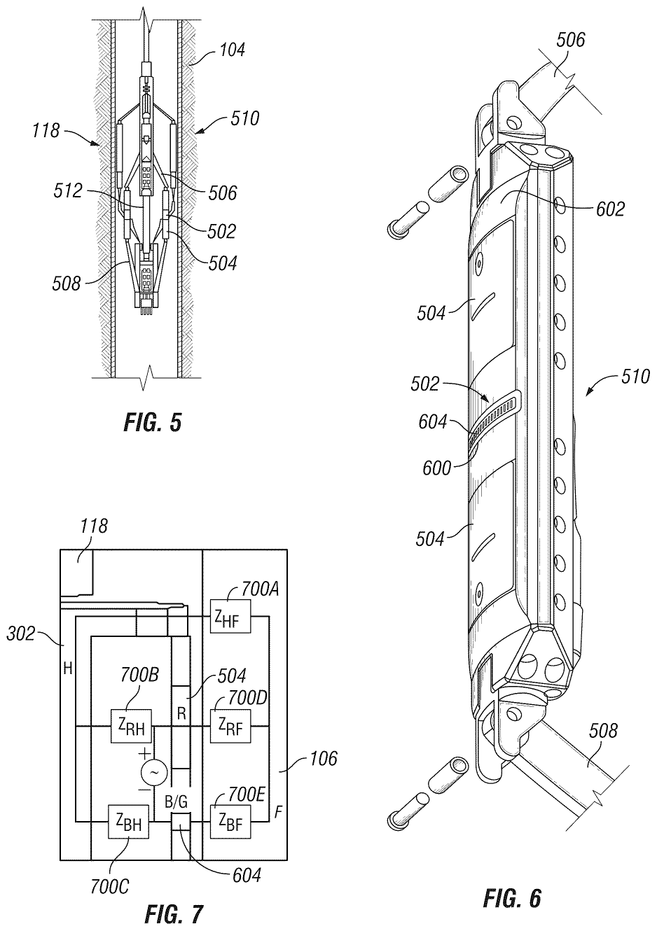

FIG. 5 illustrates an example of an imaging module 118 disposed in wellbore 104. In this example, imaging analysis module may be attached to vehicle 104. In examples, it should be noted that downhole tool 102 may not be attached to a vehicle 104 (e.g., referring to FIG. 1). Downhole tool 102 may be supported by rig 106 at surface 108. Imaging module 118 may be tethered to vehicle 104 through conveyance 102 (e.g., referring to FIG. 1). Conveyance 102 may be disposed around one or more sheave wheels 112 to vehicle 104. Conveyance 102 may include any suitable means for providing mechanical conveyance for imaging module 118, including, but not limited to, wireline, slickline, coiled tubing, pipe, drill pipe, drill string, downhole tractor, or the like. In some examples, conveyance 102 may provide mechanical suspension, as well as electrical connectivity, for imaging module 118.

Conveyance 102 may include, in some instances, a plurality of electrical conductors extending from vehicle 110. Conveyance 102 may include an inner core of seven electrical conductors covered by an insulating wrap. An inner and outer steel armor sheath may be wrapped in a helix in opposite directions around the conductors. The electrical conductors may be used for communicating power and telemetry between vehicle 110 and imaging module 118.

Conveyance 102 may lower imaging module 118 in wellbore 104. Generally, wellbore 104 may comprise horizontal, vertical, slanted, curved, and other types of borehole geometries and orientations. Imaging module 118 may be used in uncased sections of the borehole. Measurements may be made by imaging module 118 in cased sections for purposes such as calibration.

Information from imaging module 118 may be gathered and/or processed by information handling system 122. For example, signals recorded by imaging module 118 may be stored on memory and then processed by imaging module 118. The processing may be performed real-time during data acquisition or after recovery of imaging module 118. Processing may alternatively occur downhole or may occur both downhole and at surface. In some examples, signals recorded by imaging module 118 may be conducted to information handling system 122 by way of conveyance 110. Information handling system 122 may process the signals, and the information contained therein may be displayed for an operator to observe and stored for future processing and reference. Information handling system 122 may also contain an apparatus for supplying control signals and power to imaging module 118.

Imaging module 118 may include a plurality of electrodes, such as button array 502. Imaging module 118 may also include a return electrode 504. It should be noted that the plurality of electrodes disposed on button array 502 may be any suitable electrode and is should be further noted that return electrode 504 may be any suitable electrode. Button array 502 and/or return electrode 504 may be disposed on at least one pad 510 in any suitable order. For example, a pad 510 may include only button arrays 128 and/or return electrodes 130. Further, a pad 510 may include both button array 502 and return electrodes 130. Pads 510 may attach to a mandrel 512 of imaging module 118 through upper arm 506 and lower arm 508. It should be noted that mandrel 512 may be defined as the supporting structure of imaging module 118 which may act as a platform for any peripheral (e.g., upper arm 506, lower arm 508, conveyance 110, etc.) to attach to imaging module 118. Upper arm 506 and lower arm 508 may extend pad 510 away from imaging module 118. In examples, both upper arm 506 and lower arm 508 may place pad 510 in contact with borehole 124. It should be noted that there may be any suitable number of arms and/or extensions that may be used to move pad 510 away from imaging module 118 and in close proximity with borehole 124, or vice versa. In examples, when imaging module 118 is not a pad-based tool, pad 510 may be replaced with at least one sensor from imaging module 118. Pad 510 may also be considered as at least one sensor from imaging module 118.

During operations, an operator may energize an individual electrode, or any number of electrodes, of button array 502. A voltage may be applied between the electrode of button array 502 and return electrode 504. The level of the voltage may be controlled by information handling system 122. This may cause currents to be transmitted through the electrode of button array 502. It should be noted that there may be any number of currents transmitted into formation 106. These currents may travel through the mud disposed in borehole 124 and formation 106 and may reach back to return electrode 504. The amount of current emitted by each electrode may be inversely proportional to the impedance seen by the electrode. This impedance may be affected by the properties of formation 106 and the mud directly in front of each electrode of button array 502. Therefore, current emitted by each electrode may be measured and recorded in order to obtain a formation image of the resistivity of formation 106.

To produce a resistivity image of formation 106, a current may be emitted from at least one electrode from button array 502 and return to return electrode 504. In examples, current may be emitted from any transmission type electrode along imaging module 118. These two electrodes may be referred to as the current electrodes. Then, the voltage drop across a pair of the electrodes of button array 502 may be measured and used to estimate the impedance of formation 106. In these alternative implementations, button array 502 may be referred to as voltage electrodes or monitor electrodes. Proposed method may operate in any of the two designs above, any other similar oil-based mud resistivity imager tool without any limitations, or a downhole imager tool working on a separate physical principle such as an acoustic imager or a density imager. In examples, imaging module 118 may operate with additional equipment (not illustrated) on surface 108 and/or disposed in a separate well measurement system (not illustrated) to record measurements and/or values from formation 106 to render a resistivity image of formation 106.

FIG. 6 illustrates an example of pad 510. It should be noted that pad 510 may be connected to imaging module 118 (e.g., referring to FIGS. 1 and 5). Pad 510 may serve to place button array 502 and/or return electrode 504 in contact with or in close proximity to borehole 124. Pad 510 may include a button array 502, a return electrode 504, a guard 600, and a housing 602. In examples, there may be a plurality of button arrays 502. In examples, return electrode 504 and button array 502 may be individual components disposed directly on imaging module 118, with or without pad 510. Button array 502 may include an injector electrode 604, wherein injector electrode 604 may be a sensor that senses impedance of formation 106. It should be noted that injector electrode 604 may be a button array 502. There may be any suitable number of injector electrodes 604 within button array 502 that may produce a desired, predetermined current. Without limitation, the range for a suitable number of injector electrodes 604 within button array 502 may be from about one injector electrode 604 to about one hundred injector electrodes 604. For example, the range for a suitable number of injector electrodes 604 within button array 502 may be from about one injector electrode 604 to about twenty-five injector electrodes 604, from about twenty-five injector electrodes 604 to about fifty injector electrodes 604, from about fifty injector electrodes 604 to about seventy-five injector electrodes 604, or from about seventy-five injector electrodes 604 to about one hundred injector electrodes 604.

In examples, there may be a plurality of return electrodes 130. One of the return electrodes 130 may be disposed on one side of button array 502, and another one of the return electrodes 130 may be disposed on the opposite side of button array 502. These return electrodes 130 may be disposed at equal distances away from button array 502 or at varying distances from button array 502. Without limitation, the distance from the center of one of the return electrodes to the button array may be from about one inch to about one foot. In examples, a voltage difference between button array 502 and return electrodes 130 may be applied, which may cause currents to be emitted from button array 502 into the mud (not illustrated) and formation 106 (referring to FIG. 1).

During operations, an operator may energize button array 502. A voltage may be applied between each injector electrode 604 and return electrode 504. The level of the voltage may be controlled by information handling system 122. This may cause currents to be transmitted through button array 502. These currents may travel through the mud and formation 106 and may reach back to return electrode 504. The amount of current emitted by each injector electrode 604 may be inversely proportional to the impedance seen by that injector electrode 604. This impedance may be affected by the properties of formation 106 and the mud directly in front of each injector electrode 604. Therefore, current emitted by each injector electrode 604 may be measured and recorded in order to obtain an image of the resistivity of formation 106.

In examples, a current may be transmitted from injector electrode 604 and return to return electrode 504. These two electrodes may be referred to as the current electrodes. Then, the voltage drops across button array 502 may be measured and used to estimate the impedance of formation 106. In these alternative implementations, electrodes of button array 502 may be referred to as voltage electrodes or monitor electrodes. Proposed method may operate in any of the two designs above or any other similar oil-based mud or water-based mud resistivity imager tool without any limitations. In the rest of the text, the imager tool will be assumed to be of the first design without any loss of generality.

Returning back to FIG. 6, guard 600 may help to focus most of the current produced by button array 502 into formation 106 radially. Guard 600 may be disposed around button array 502. Guard 600 may include the same potential as button array 502.

In examples, housing 602 may serve to protect button array 502 and return electrodes 130 from the surrounding mud and formation 106. Housing may be made with any suitable material. Without limitation, suitable material may include metals, nonmetals, plastics, ceramics, composites and/or combinations thereof. In examples, housing 602 may be a metal plate. Housing 602 may be connected through upper arm 506 to imaging module 118 (e.g., referring to FIG. 1). An insulating material may be used to fill the remaining portions of pad 510. In examples, ceramics may be used as the insulating material to fill the remaining portions of pad 510.

An impedance value may be calculated through the current transmitting between an injector electrode 604 and formation 106 for each injector electrode 604. The voltage between button array 502 and return electrodes 130 may be measured and divided by the transmitted current to produce a value for the impedance seen by each injector electrode 604. Most of the transmitted current may be returned to return electrodes 130 although some portions of it may return through housing 602 and imaging module 118 (e.g., referring to FIG. 1).

During logging operations, measurement data taken by pad 510 may include effects of resistivity and permittivity. Measurements may contain contributions from oil-based mud that is may be disposed between pad 510 and the wall of borehole 124 as well as the signal coming from the formation. As such, imaging module 118 may be configured to acquire traditional downhole measurements as a downhole measurement log, utilizing a pad-based tool which may record the resistivity of the rock formation, an acoustic scanning tool which may read the acoustic reflectivity of the rock formation, a neutral density imager, an optical imager, any possible downhole tools and/or the like. Imaging module 118 may be a water-based mud resistivity imager tool or an oil-based resistivity imager tool.

In general, the measurement medium of pad 510 may be modeled as a homogeneous formation with a thin layer of oil-based mud between pad 510 and formation 106. When pad 510 is placed on formation 106 without a mud layer, response measurement may only be from formation 106. However, when there is a mud layer present, the response is influenced by the thickness of the mud layer as well as the mud properties, in addition to the properties of formation 106 behind the mud layer. The response for certain formation 106 predominantly consists of the mud signal, which may make this response suitable for determining mud properties. Imaging module 118 may be disposed in a drilling system 300 in wellbore 104.

FIG. 7 illustrates an example of a circuit model that may approximate the pad 510 illustrated in FIG. 2. Effects of the transmitted current may be approximately characterized by a housing-to-formation impedance value 700A, a return electrode-to-housing impedance value 700B, a return electrode-to-formation impedance value 700C, a button-to-housing impedance value 700D, and a button-to-formation impedance value 700E. Impedance may be calculated below, wherein Z is the impedance, V_{BR} is the button-to-return electrode voltage and I_{B} is the button current:

$$Z = \frac{{V}_{BR}}{{I}_{B}} \qquad ( 1 )$$

The value calculated in Equation (1) may be equal to Z_{BF}+Z_{RF}, as shown in FIG. 7, wherein Z_{BF} is the impedance from injector electrode 604 to formation 106 and Z_{RF} is the impedance of return electrode 130 to formation 106. Note that for different injector electrodes 204 of the button array 502, these impedances may differ based on the variations in borehole 124 (e.g., referring to FIGS. 1 and 2) and the environment. These variations in measured impedances in an impedance image may be used to determine geophysical features. Z Also note that both Z_{BF} and Z_{RF} have contributions from both the surrounding mud and formation 106 (e.g., referring to FIG. 1). Thus, equivalently it can be written in Equation (2) as:

$$Z \approx {Z}_{BF} {Z}_{BF} = {Z}_{mud} {Z}_{BF} {Z}_{BF} = {Z}_{mud} {Z}_{BF} {Z}_{BF} = {Z}_{mud} {Z}_{BF} {Z}_{BF} = {Z}_{mud} Z \approx {Z}_{BF} {Z}_{BF} = {Z}_{mud} {Z}_{BF} {Z}_{BF} = {Z}_{mud} {Z}_{BF} {Z}_{BF} = {Z}_{mud} {Z}_{BF} {Z}_{BF} = {Z}_{mud} + {Z}_{F} \qquad ( 2 )$$

As a result, measured impedance may have contributions from both the mud and formation 106, wherein Z_{mud} is the impedance of the mud and Z_{F} is the impedance of formation 106. Imaginary parts of Z_{F} and Z_{mud} may be assumed to be mainly capacitive. Assuming this capacitance may be in parallel with the resistive portion, then Z_{BF} may also be written as:

$${Z}_{BF} = \frac{1}{( \frac{1}{{R}_{M}} + j ω {C}_{M} )} + \frac{1}{( \frac{1}{{R}_{F}} + j ω {C}_{F} )} \qquad ( 3 )$$

wherein R_{M} is the mud resistance, R_{F} is the resistance of formation 106, C_{M} is the mud capacitance, C_{F} is the capacitance of formation 106, j is the unit imaginary number, and w is the angular frequency. Both the mud resistance and mud capacitance may increase as standoff increases and may decrease with the increase in effective area of injector electrode 604. “Standoff” may be used to denote the distance of the pad 510 (e.g., Referring to FIG. 5) from a wall of wellbore 104 (e.g., referring to FIG. 1). Standoff of each injector electrode 604 in button array 502 may vary. In examples, standoffs of return electrode 130 may differ from those of injector electrodes 604 as well. Standoff variations may significantly affect button-to-formation impedance value 700E. In the simplified circuit model, it may be assumed that the standoff of each component of pad 510 may be constant. Standoff may assume that pad 510 is movable while imaging module 118 remains immobile. In examples, to achieve large distances from the wall of wellbore 104, imaging module 118 may be moved along with pad 510. In examples, the term “eccentricity” may be used instead of “standoff”.

Equation (3) may be used to obtain basic performance curves for imaging module 118. In examples, this may be applicable only for pad-based tools and other downhole tools may rely on their own processing and mathematical techniques. The same formulas may hold for water-based mud resistivity imagers, but Z_{BF} would be ~Rf since frequency (omega) is very low. Other imagers do not measure resistivity. Although there exist higher order effects that would not be captured in such a simple model, in most practical cases the circuit model may be used successfully to gain valuable intuition. In the case there is no mud (i.e. no standoff), Equation (3) can be modified into Equation (4):

$${Z}_{BF} = \frac{{R}_{F}}{( 1 + j ω {C}_{F} {R}_{F} )} = \frac{{R}_{F} - j ω {{C}_{F} ( {R}_{F} )}^{2}}{( 1 + {( ω {C}_{F} {R}_{F} )}^{2} )} \qquad ( 4 )$$

As a result, when formation resistivity is low, real part of the measured impedance would be approximately equal to R_{F}. This resistance is in turn a function of resistivity. In most cases, this function may be approximated as a simple constant multiplying the formation resistivity, which may be denoted as the tool constant k which is a function of the tool geometry:

$${R}_{F} \approx k {ρ}_{F} \qquad ( 5 )$$

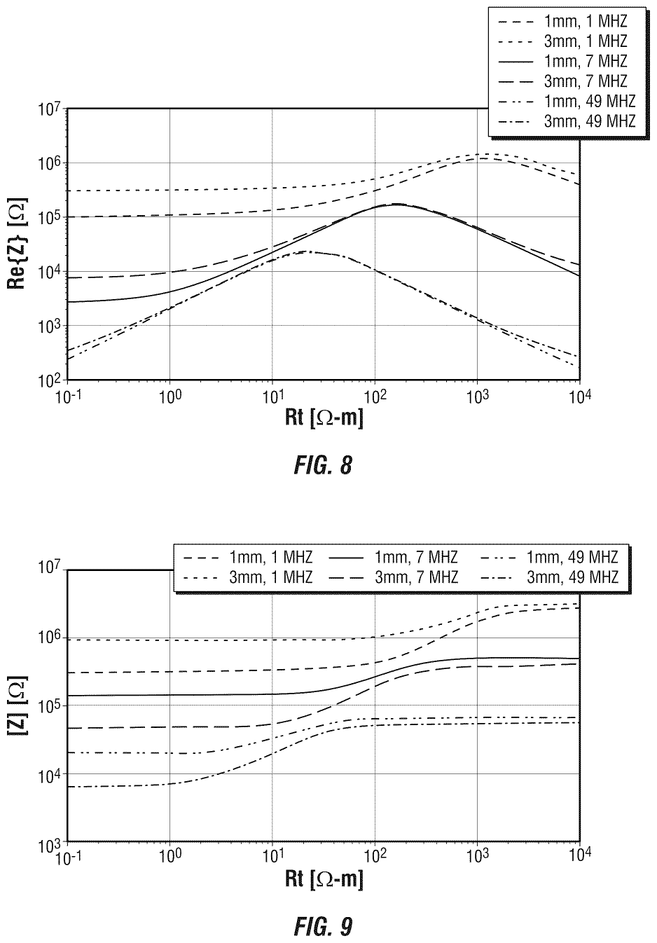

These basic performance curves may be fairly accurate in homogeneous formations 106 (e.g., referring to FIG. 1) in determining the variation of the response of an exemplary injector electrode 604 (e.g., referring to FIG. 6) in button array 502 with changing environmental parameters. In FIG. 8, the real part of the measured impedance versus the formation resistivity may be determined using Equation (3), which is illustrated on graph in FIG. 8. The imaginary part of the impedance may be determined by the mud capacitance, therefore it may not be necessary to plot it. In an example, illustrated in FIG. 9, it may be assumed that formation permittivity (εF) is 15, mud permittivity (εM) is 6, and mud resistivity (ρM) is 8000 Ω-m. Results for three different frequencies (1 MHz, 7 MHz and 49 MHz) at two different standoffs (so=1 mm and so=3 mm), where (so) stands for standoff of the tool, may be displayed in FIG. 8.

As illustrated in FIG. 8, a separation between different standoffs at lower formation resistivities may be viewed. This effect may be more pronounced if the frequency is lower. At higher formation resistivities, the dielectric effect in formation 106 (e.g., referring to FIG. 1) may cause a roll-off in measured impedance, as illustrated in FIG. 8. Operating in a linear region of the curve, displayed in FIG. 8, may produce a more accurate correspondence between the impedance image and that of the true formation resistivity. The standoff effect at low formation resistivities may cause an ambiguity in the interpretation of the impedance images. These raw measurements may be used, but the contrast of the resistivity image may be reduced. Furthermore, small errors in standoff measurements may cause a large difference in the impedance reading. It may be observed from FIG. 5 that measured impedance may begin to decrease as the formation resistivity increases. This “rolloff” may be caused by the dielectric effects in the formation 106 (e.g., referring to FIG. 1) and may become more pronounced at higher frequencies.

The graph in FIG. 8 illustrates that lower frequencies may be more suitable for measuring high formation resistivities while higher frequencies are more suitable to measure lower formation resistivities. For this reason, when the imaging module 118, is an oil-based mud imager tool, it may generally be implemented as a multi-frequency tool. Multi-frequency measurements may also reduce uncertainty in resolving different mud and formation properties through an inversion or machine learning process. Operational frequencies of imaging module 118 (e.g., referring to FIGS. 1 and 3) may be adjustable through a central control unit and may be changed based on the specifications of the job.

The graph in FIG. 9 illustrates an absolute value of the impedance versus the formation resistivity for the same case in FIG. 9. As illustrates, the absolute value of the impedance does not suffer a “roll-off” due to the dielectric effect at high formation resistivities although the sensitivity of imaging module 118 (e.g., referring to FIG. 1) to the resistivity is reduced. For example, the absolute value is almost flat with changing formation resistivity.

Results from the graphs of FIGS. 8 and 9 of a simplified circuit approximation illustrate that the impedance measured by an oil based mud imager tool may not accurately reflect the variations in formation resistivity due to the effects of formation permittivity, mud resistivity and mud permittivity. Currently, inversion based approaches have been used to determine the formation resistivity (along with other formation and mud parameters) from measurements. These model-based inversion techniques are based on simulating the response of imaging module 118 (e.g., referring to FIG. 4) using a forward model. For example, a forward model may be obtained with a 3D electromagnetic simulation software. Then, the parameters that minimize the difference between the measurements and the model response corresponding to these parameters are returned as the inversion output. In examples, an iterative process may be used for this purpose, such as the Gauss-Newton method. Depending on the accuracy of the forward model used in inversion, inversion results may vary in accuracy. Additionally, it may be beneficial to apply regularization and use known relationships between inverted parameters in an inversion approach.

The analysis based on a simple circuit-based model in the previous section demonstrated the combined effects of formation permittivity and formation resistivity along with mud's contribution meant a complicated response is obtained by an oil-based mud resistivity imaging module 118 (e.g., referring to FIG. 1) which, in most instances, is not directly proportional to formation resistivity. Furthermore, higher order effects from causes such as tool geometry and materials used in tool's construction, along with the nonlinearities brought forth by tool electronics may further affect imaging module 118 when it is disposed within wellbore 102. Although measurements made using mostly homogeneous media such as test tanks may help with the tool characterization, it is desirable to analyze the tool behavior in the presence of an actual formation sample, in particular, imaging module 118 response in the presence of fine features such as fractures are of interest. Users of such tools are particularly interested in how open and closed fractures are being observed in images which in turn affect their analysis of the permeability of the formation. However, confidence of these measurements are limited when operating imaging module 118 within wellbore 102. Thus, core sample 400 may be measured by imaging module 118 at surface 112 to validate results. This may provide a baseline for more confidence in measurements.

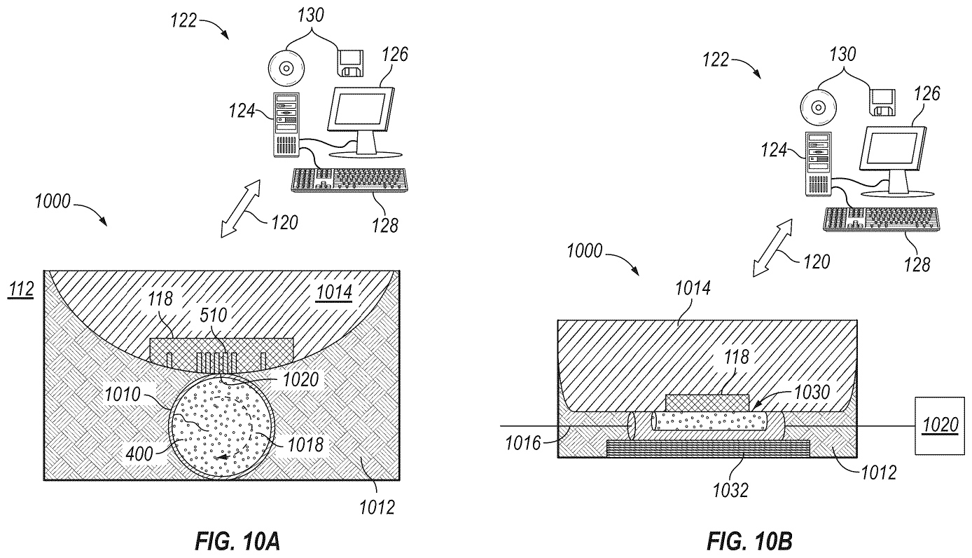

FIG. 10A illustrates rotating shaft example 1000 for utilizing imaging module 118 on core sample 400. FIG. 10B illustrates a side view of rotating shaft example 1000 for utilizing imaging module 118 on core sample 400. In examples, imaging module 118 and rotating shaft example 1000 may be connected to and controlled by information handling system 122, such that information handling system may record measurements and operate all components via commands. In rotating shaft example 1000, core sample 400 may be located inside a nether section of lower second container 1010 and may be cylindrical in shape. In examples, a latching mechanism between core storage module 314 (e.g., referring to FIG. 3) and lower second container 1010 of the apparatus may be manufactured to pressurize such that the loss of formation fluid in core sample 400 is minimized when transporting core sample 400 to surface 112. As discussed above, core storage module 314 (e.g., referring to FIG. 3) may be a pressurized vessel to ensure the core sample remains saturated with original formation fluid. Core module 314 may then be utilized directly within rotating shaft example 1000 as lower second container 1010. In other examples, core sample 400 may be transported to lower second container 1010 via a pressurized vessel. Additionally, core sample 400 may be transported without being pressurized, where downhole conditions are then recreated within lower second container 1010.

Herein, surface 112 may be defined as any environment, not within wellbore 102. Within lower second container 1010, formation core may be held within a rotating shaft 1016 causing a rotation 1018. Rotating shaft 1016 may have adjustable ends that may hold core sample 400 using a tension mechanism. In other examples, rotating shaft 1016 may penetrate the ends of core sample to hold core sample 400. Further, rotating shaft 1016 may lead to outside lower second container 1010 and may be connected to motor 1020 that enables precise rotation 1018 of core sample 400. In other examples, rotating shaft 1016 may be connected to a crank (not illustrated) that may be rotated manually.

Lower second container 1010 may comprise opening 1030 to another larger, upper third container 1014 that may be semi-cylindrical in shape. Upper third container 1014 may be a semi-circle or any other feasible shape to allow imaging contact between imaging module 118 and core sample 400. Lower second container 1010 and upper third container 1014 may be filled with a fluid that is representative of a drilling mud. These two sections may be pressurized to levels similar to or the same as what would be experienced downhole in wellbore 102 (e.g., referring to FIG. 1). Imaging module 118 may be comprised within upper third container 1014. Imaging module 118 may comprise pads 510 (e.g., referring to FIG. 5). However, this is one example and other types of imaging module 118 may comprise a pad-based tool which may record the resistivity of the rock formation, an acoustic scanning tool which may read the acoustic reflectivity of the rock formation, a neutral density imager, an optical imager, any possible downhole tools and/or the like. In this example, pads 510 do not need to be fully submerged and in some embodiments, parts of the pad may not lie within the mud. However, the actual sensor used for making measurements (such as at least one of the button array 502 on one of the pads 510 for imaging module 118) should lie within the mud for proper operation. In examples, the rest of imaging module 118 (such as the arm 506 connecting the pad) may not be comprised and the single pad may be directly connected to electronic circuitry that enables transmission of the signals and measuring of the tool response.

In examples pad 510 may be located such that a sensor of the tool substantially coincides with the opening between the upper and lower sections. For example, imaging module 118 may coincide with at least one electrode within button array 502 (e.g., referring to FIG. 5) such that the metallic surface of the electrode may substantially contained within opening 530. Thus, at least one electrode from within button array 502 may touch or have sufficient exposure to core sample 400 below or may be in close proximity to core sample below with the distance (i.e., standoff) between the electrode and core sample 400 filled with the representative mud. In examples, a small standoff (between 0.01 mm-1 mm) between core sample and the electrode is operable in order to prevent abrasion of the core. On and off touching of the core by the tool would also cause sharp changes in the tool response. In examples, maintaining the standoff constant during operations may be possible. This may be accomplished by making the support structure 1032 for the core to consist of thin layers of a rigid or flexible material and adjusting the number of such layers to obtain the desired standoff. In general, standoff between the core sample and the at least one sensor of the imaging module is kept substantially constant during a set of measurements. Herein, substantially constant is defined as not more than a 5% deviation from its magnitude and/or direction. In examples, support structure 1032 may be configured to move or adjust. As such, measurements may be repeated with different standoffs (between 0.001 mm-0.1 mm, 0.1 mm-1 mm, 1 mm-10 cm, or 10 cm-10 meters) to characterize the affect of standoff. As such, at least one sensor (or pad 510), with rotation 1018 may produce a core sample image with an azimuthal coverage.

In examples, opening 1030 between the upper third container 1014 and lower second container 1010 may be in the form of a linear slot rather than a small circular one slot. As such, pad 510 may have additional exposure to core sample 400. However, this is not absolutely needed since the large area of pad 510 has little impact on the measured impedance. Nevertheless, a first container 1012 may be at least partially filled with a fluid that approximates the average resistivity of the core sample 400. The first container 1012, may be insulated and large enough to make the effect of rotating shaft example 1000 negligible. For imaging module 118, due to the high frequencies utilized, third container 1014 may be a relatively small container with dimensions in the order of a couple of feet may be enough for this purpose. A plastic film (not illustrated) may separate portions filled with the fluid representing mud and the fluid representing the formation. In other examples, first container 1012 may be substantially filled with drilling mud as explained below.

FIGS. 10A and 10B are one example and other examples may be applicable as well. First container 1012, upper third container 1014, and lower second container 1010 may be in any possible shape. For example, second container 1010 may be replaced with a rectangular prism while upper third container 1014 may instead be semi-spherical. In addition, the upper and lower nature of the sections may be flipped or laid side by side. Furthermore, upper third container 1014 and lower second container 1010 may be manufactured together and may be supported by the walls of first container 1012. In other examples, they may be connected by mechanisms such as screws and they may include additional support structures to hold them in place.

As such, rotating shaft example 1000 with imaging module 118 may comprise one or more pads from a downhole imaging tool that is commonly used to image formation in borehole 124 (e.g., referring to FIG. 1). This may be accomplished by separating one or more pad(s) 510 from downhole tool 100 and fitting pad 510 in an up-hole surface fixture for imaging core samples 400 (e.g., referring to FIG. 10) on surface 112. In other examples, the same analysis yielded by rotating shaft example 1000 may be achieved downhole, possibly within borehole 124. To illustrate, downhole tool 100 may be modified to comprise some or all components of rotating shaft example 1000 to produce a core sample image with an azimuthal coverage. The implementation downhole may vary. For example, instead of pad 510, a return electrode 504 (e.g., referring to FIG. 6) and/or button array 502 (or a single electrode may be utilized. In addition, there may be rotation 1018 applied to core sample 400 in some mechanical mechanism within downhole tool 100.

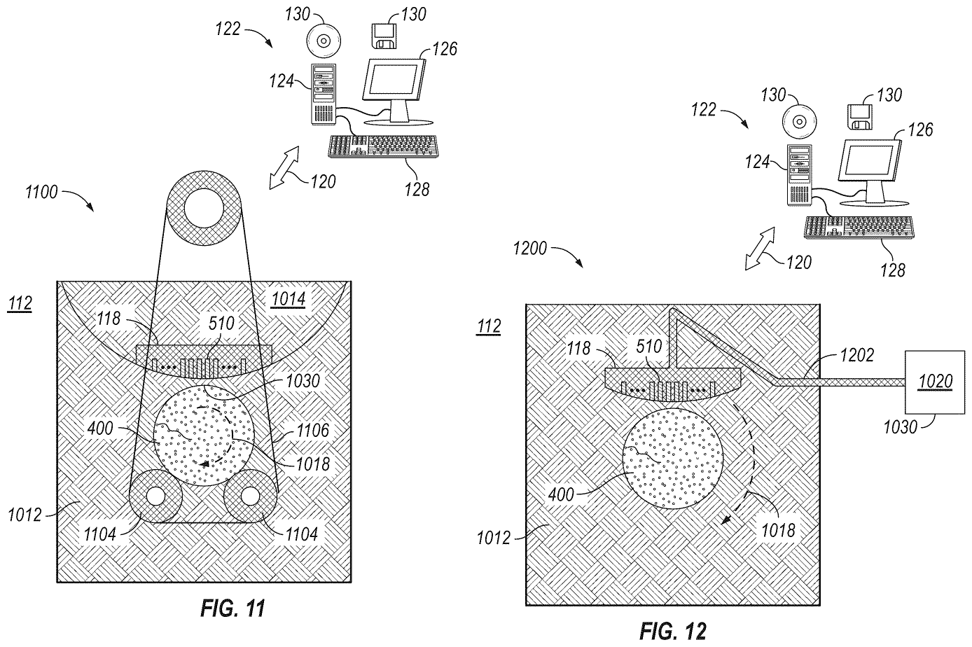

FIG. 11 illustrates pulley example 1100 for utilizing imaging module 118 on core sample 400. In examples, imaging module 118 and pulley example 1100 may be connected to and controlled by information handling system 122, such that information handling system may record measurements and operate all components via commands. In this example core sample 400 and pad 510 may be located in a pressurized container filled with just a fluid representative of the mud. Upper third container 1014 may still be utilized to provide support to pad 510 and may be separated from first container 1012 with a plastic film comprising opening 1030. However, this plastic film may not need to totally separate upper third container 1014 from first container 1012 and it may be held using supporting columns. In pulley example 1100, core sample 400 may be held using rotating core supports 1104. Core supports 1004 may be connected with belt 1106 which in turn is driven by stepper motor 1102 producing rotation 1018. As such, at least one sensor (or pad 510), with rotation 1018 may produce an azimuthal coverage of core sample 400.

Core supports 1004 may be held on mounts (not illustrated). In addition, a manual rotation of the belt using a crank is also possible. Either core sample 400 itself or the rotating core supports 1104 may be moved laterally to enable axial logging of core sample 400. In this example, a return electrode from pad 510 would mostly be covered with highly resistive mud. However, due to the large cross-sectional area of the return electrode, measurements would qualitatively still be similar to what is observed with apparatus shown in 10A and 10B although quantitative differences would be observed. Another example utilizing hydraulics may also be possible.

FIG. 12 illustrates hydraulic arms example 1200 for utilizing imaging module 118 on core sample 400. In examples, imaging module 118 and hydraulic arms example 1200 may be connected to and controlled by information handling system 122, such that information handling system may record measurements and operate all components via commands. Core sample 400 may be held stationary and imaging module 118 be rotated via hydraulic arms 1202 driven by motor 1020. Hydraulic arms and motor 1020 may support rotation 1018 and lateral movement of pad 510. As such, at least one sensor (or pad 510), with rotation 1018 may produce an azimuthal coverage of core sample 400. Both core sample 400 and imaging module 118 may be in first container 1012 filled with a fluid representative of the mud. There may be mounts (not illustrated) to hold core sample 400 in place. In other examples, core sample 400 may be suspended from its ends using cables. Rotating shaft example 1000, pulley example 1100, and hydraulic arms example 1200 are examples for measuring and/or imaging core sample 400 on surface 112 and may be applied in a workflow below. In addition, a combination or other example of rotating shaft example 1000, pulley example 1100, and hydraulic arms example 1200 may be possible as well.

Once core sample 400 is obtained, it may also be imaged up hole through traditional techniques. In examples, known properties of the core sample may be obtained by traditional techniques such as, visually inspecting core sample 400, hyperspectral imaging using a hyperspectral camera, X-ray fluorescence spectroscopy, Laser-Induced Breakdown Spectroscopy (LIBS), measuring core sample length, diameter, width, porosity, permeability, grain size analysis, density, and/or wettability. These devices may, in some instances, be handheld. In examples, core sample 400 may be rotated to obtain 360° images of core sample 400. These images may be compared with the image obtained through the imaging module.

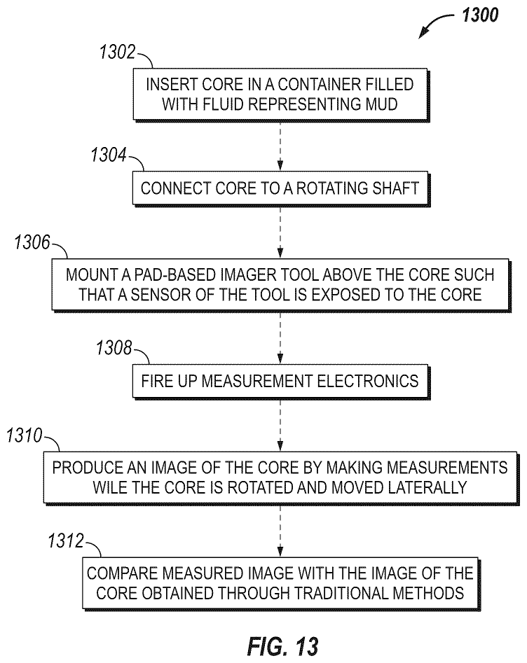

FIG. 13 illustrates a workflow for obtaining measurements using either rotating shaft example 1000, pulley example 1100, and/or hydraulic arms example 1200. In block 1302, core sample 400 (e.g., referring to FIG. 10A) may be positioned within a first container 1012 containing fluid representing the drilling mud. In block 1304, a core sample 400 may be connected to a rotating shaft such that when the shaft is rotated, core is rotated synchronously. In block 1306, an imaging module 118 comprising a pad-based imager tool is mounted above core sample and at least one sensor of the tool is exposed to core sample 400. In block 1308, imaging module 118 may begin acquiring measurements. In block 1310, while core sample 400 is rotated and moved to produce a core image log with imaging module 118. In block 1312, the core image log may be compared with a downhole measurement log obtained using traditional methods, described above to characterize and/or validate imaging module 118 within wellbore 102. Imaging parameters returned by the machine learning in block 1312 are then returned in block 1310 for physical or software modeling adjustments, as discussed below. In block 1312 these parameters may either be visualized as an image on a video display 126 (e.g., referring to FIG. 1), and/or may be used as inputs for other algorithms on information handling system 122. Herein characterizing may be defining imaging module 118 full capabilities, limitations, and performance metric. Herein, validating may be ensuring imaging module 118 provides reliable and accurate measurements.

In examples, characterization and validation may involve the processed answer products of imaging module 118. For example, an inversion routine may be applied to imaging module 118 response as it is known in the art to obtain images of formation resistivity, formation permittivity and standoff. These may then be used to characterize formation features. In one example, formation feature may be a fracture, and the imager answer products may be used to estimate whether the fracture is closed or not. This estimate may then be validated with the traditional measurements of core sample 400 and known properties of core sample 400. Determining known properties of the core sample may be performed by visual inspection, hyperspectral imaging, X-ray fluorescence spectroscopy, or Laser-Induced Breakdown Spectroscopy. If there is a mismatch, physical or software modeling adjustments may be applied to improve imaging module 118 results after logging or to the actual functioning of imaging module 118. Such physical or software modeling adjustments may involve adjusting calibration of the pad-based imager tool, adjusting the model that is used to simulate the pad-based imager tool response to obtain the answer products, or adjusting the parameters of the answer products (e.g., inversion parameters). Herein, answer products may be aperture of any fissure in the well bore which may include (but not limited to) natural fracture aperture, induced fracture aperture, borehole breakout width, and/or maximum vug width at the borehole wall, formation resistivity, permittivity and standoff images in the case of inversion. In addition, answer products may be extended to detecting stratigraphic horizons (such as unconformities), soft sediment deformation, bedform structure. Answer products may be utilized for adjusting inversion parameters to match the answer products to what is observed through traditional methods on core sample 400.

In examples, the answer products are obtained through an inversion scheme, a machine learning scheme, or a hybrid inversion/machine-learning scheme. In other examples, machine learning based methods or hybrid machine learning/inversion-based methods may be used to obtain aforementioned answer products. In other examples, answer products may be obtained and compared with the core image. These may include, without limitation, a mud effect corrected image, a dielectric effect corrected image, and/or an image obtained by blending data from different frequencies/data sources. The obtained answer products are used to determine whether a fracture in core sample 400 is open or closed.

Improvements in the application over current technology may be demonstrated herein. For example, existing tools for core imaging are not designed for downhole operation. Conversely, imaging of cores using downhole imaging tools is not available. Pad-based imaging tools require the imaging tool to be in close proximity to the core and this distance to be accurately controlled. Existing techniques do not have such limitations and as such do not have the means to control and adjust the distance accurately. To replicate downhole conditions, core sample and the downhole tool should be submerged in fluids representing mud and in some cases formation. This is not required and performed with existing techniques. Furthermore, a technique for making measurements on a pressurized container to determine properties of the formation fluids and rock is described. The existing test instruments are not built for the purpose of formation testing. Core samples and the testing equipment are submerged in drilling mud and/or other fluids. Core samples and/or testing equipment are moved or rotated during measurements to obtain core images.

The various systems, apparatus, methods, and other constructs may include any suitable combination of the features disclosed herein, including one or more of the following statements.

Statement 1. A system comprising: a first container at least partially filled with a fluid; a core sample disposed within the first container and at least partially submerged within the fluid; and at least one sensor from an imaging module submerged within the fluid disposed at a standoff from the core sample, wherein the at least one sensor is configured to obtain a core sample image while the at least one sensor of the imaging module is rotated or moved around the core sample.

Statement 2. The system of statement 1, wherein the core sample is disposed within a lower second container within the first container.

Statement 3. The system of statement 2, the imaging module is disposed within an upper third container within the first container.

Statement 4. The system of statement 3, further comprising an opening on the upper third container and the lower second container, wherein at least a sensor of the of the imaging module is located within the opening.

Statement 5. The system of statement 1, wherein the core sample is obtained utilizing a pressurized vessel to ensure the core sample remains saturated with original formation fluid.

Statement 6. The system of statement 1, wherein the mineral fillers improve thermal conductivity, heat resistance, and thermal barrier capabilities of the acrylic polymer-based elastomer.

Statement 7. The system of statement 6, wherein the support structure is further configured to adjust the standoff between the core sample and the at least one sensor.

Statement 8. The system of statement 1, further comprising a rotating shaft configured to rotate the core sample utilizing a motor or manually by turning a crank.

Statement 9. The system of statement 8, wherein the rotating shaft holds the core sample through tension or penetrates the core sample to enable rotation.

Statement 10. The system of statement 1, wherein the core sample is held on rotating core supports and rotated by a belt utilizing a motor or manually by turning a crank.

Statement 11. The system of statement 1, wherein the imaging module is held using one or more hydraulic arms and rotated around the core sample utilizing a motor or manually by turning a crank.

Statement 12. The system of statement 1, wherein the imaging module is a water-based mud resistivity imager tool or an oil-based resistivity imager tool.

Statement 13. The system of statement 1, wherein the core sample image comprises an azimuthal coverage of the core sample.

Statement 14. A method comprising: forming a core sample image of a core sample in a first container at least partially filled with a fluid utilizing an imaging module, wherein the first container comprises: at least one sensor from the imaging module is submerged within the fluid and disposed at a standoff from the core sample; and acquiring a downhole measurement log with the imaging module.

Statement 15. The method of statement 14: further comprising characterizing the downhole measurement log of the imaging module.

Statement 16. The method of statement 14, further comprising validating the downhole measurement log of the imaging module.

Statement 17. The method of statement 16, wherein validating is performed by comparing the core sample image with known properties of the core sample obtained by traditional techniques.

Statement 18. The method of statement 17, further comprising determining known properties of the core sample by traditional techniques comprising visually inspecting the core sample, hyperspectral imaging using a hyperspectral camera, X-ray fluorescence spectroscopy, Laser-Induced Breakdown Spectroscopy (LIBS), length, diameter, width, porosity, permeability, grain size analysis, density, and/or wettability.

Statement 19. The method of statement 14, further comprising determining answer products through an inversion scheme, a machine learning scheme, or a hybrid inversion/machine-learning scheme.

Statement 20. The method of statement 19, further comprising determining whether a fracture of the core sample is open or closed using the answer products.

Although the present disclosure and its advantages have been described in detail, it should be understood that various changes, substitutions and alterations may be made herein without departing from the spirit and scope of the disclosure as defined by the appended claims. The preceding description provides various examples of the systems and methods of use disclosed herein which may contain different method steps and alternative combinations of components. It should be understood that, although individual examples may be discussed herein, the present disclosure covers all combinations of the disclosed examples, including, without limitation, the different component combinations, method step combinations, and properties of the system. It should be understood that the compositions and methods are described in terms of “comprising,” “containing,” or “including” various components or steps, the compositions and methods can also “consist essentially of” or “consist of” the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present examples are well adapted to attain the ends and advantages mentioned as well as those that are inherent therein. The particular examples disclosed above are illustrative only, and may be modified and practiced in different but equivalent manners apparent to those skilled in the art having the benefit of the teachings herein. Although individual examples are discussed, the disclosure covers all combinations of all of the examples. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative examples disclosed above may be altered or modified and all such variations are considered within the scope and spirit of those examples. If there is any conflict in the usages of a word or term in this specification and one or more patent(s) or other documents that may be incorporated herein by reference, the definitions that are consistent with this specification should be adopted.

What is claimed is:

1. A system comprising:
a first container at least partially filled with a fluid;
a core sample disposed within the first container and at least partially submerged within the fluid; and
at least one sensor from an imaging module submerged within the fluid disposed at a standoff from the core sample, wherein the at least one sensor is configured to obtain:
a core sample image while the at least one sensor of the imaging module is rotated or moved around the core sample.

2. The system of claim 1, wherein the core sample is disposed within a lower second container within the first container.

3. The system of claim 2, wherein the imaging module is disposed within an upper third container within the first container.

4. The system of claim 3, further comprising an opening on the upper third container and the lower second container, wherein at least a sensor of the of the imaging module is located within the opening.

5. The system of claim 1, wherein the core sample is obtained utilizing a pressurized vessel to ensure the core sample remains saturated with original formation fluid.

6. The system of claim 1, further comprising a support structure configured to maintain a standoff substantially constant between the core sample and the at least one sensor.

7. The system of claim 6, wherein the support structure is further configured to adjust the standoff between the core sample and the at least one sensor.

8. The system of claim 1, further comprising a rotating shaft configured to rotate the core sample utilizing a motor or manually by turning a crank.

9. The system of claim 8, wherein the rotating shaft holds the core sample through tension or penetrates the core sample to enable rotation.

10. The system of claim 1, wherein the core sample is held on rotating core supports and rotated by a belt utilizing a motor or manually by turning a crank.

11. The system of claim 1, wherein the imaging module is held using one or more hydraulic arms and rotated around the core sample utilizing a motor or manually by turning a crank.

12. The system of claim 1, wherein the imaging module is a water-based mud resistivity imager tool or an oil-based resistivity imager tool.

13. The system of claim 1, wherein the core sample image comprises an azimuthal coverage of the core sample.

14. A method comprising:
forming a core sample image of a core sample in a first container at least partially filled with a fluid utilizing an imaging module, wherein the first container comprises:
at least one sensor from the imaging module is submerged within the fluid and disposed at a standoff from the core sample; and
acquiring a downhole measurement log with the imaging module.

15. The method of claim 14, further comprising characterizing the downhole measurement log of the imaging module.

16. The method of claim 14, further comprising validating the downhole measurement log of the imaging module.

17. The method of claim 16, wherein validating is performed by comparing the core sample image with known properties of the core sample obtained by traditional techniques.

18. The method of claim 17, further comprising determining known properties of the core sample by traditional techniques comprising visually inspecting the core sample, hyperspectral imaging using a hyperspectral camera, X-ray fluorescence spectroscopy, Laser-Induced Breakdown Spectroscopy (LIBS), length, diameter, width, porosity, permeability, grain size analysis, density, and/or wettability.

19. The method of claim 14, further comprising determining answer products through an inversion scheme, a machine learning scheme, or a hybrid inversion/machine-learning scheme.

20. The method of claim 19, further comprising determining whether a fracture of the core sample is open or closed using the answer products.
